(19) United States
(12) Patent Application Publication
(10) Pub. No.: US 2019/0062332 A1
(43) Pub. Date: Feb. 28, 2019

(54) PROCESS FOR THE PREPARATION OF LEDIPASVIR AND INTERMEDIATES THEREOF

(71) Applicant: Lupin Limited, Mumbai (IN)

(72) Inventors: Swapnil Sudhakar Deshmukh, Pune (IN); Manoj Kunjabihari Agrawal, Pune (IN); Adinath Murlidhar Jain, Pune (IN); Himanshu Madhav Godbole, Pune (IN); Girij Pal Singh, Pune (IN)

(21) Appl. No.: 16/073,606

(22) PCT Filed: Jan. 28, 2017
(86) PCT No.: PCT/IB2017/050464

§ 371 Date: Jul. 27, 2018

(30) Foreign Application Priority Data
Feb. 1, 2016 (IN) 201621003558
Nov. 9, 2016 (IN) 201621038343

(51) Int. Cl. C07D 471/08; C07D 403/04

(52) U.S. Cl. CPC C07D 471/08; C07D 403/04

(57) ABSTRACT

The present invention relates to process for preparation of ledipasvir of formula 1 and its novel intermediates. The process involves reaction of compound of formula 2 with compound of formula 3 to yield a compound of formula 4, deprotection of compound of formula 4 to yield compound of formula 5 and conversion of compound of formula 5 to Ledipasvir wherein PG is an amine protecting group provided that amino protecting group is not carbomethyloxy (—COOCH3) group; X and Y are leaving groups.

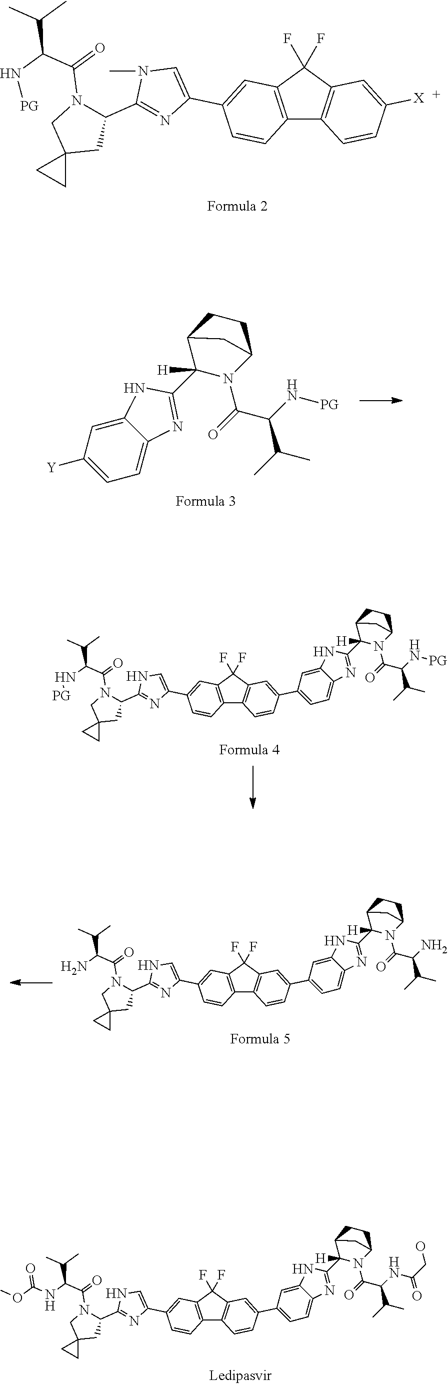

### FIELD OF THE INVENTION

The present invention relates to a process for the preparation of anti-HCV compound Ledipasvir, having the chemical name (1-{3-[6-(9,9-difluoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methylpropyl)-carbamic acid methyl ester by using novel intermediates.

### BACKGROUND OF THE INVENTION

Hepatitis C is recognized as a chronic viral disease of the liver which is characterized by liver disease. Although drugs targeting the liver are in wide use and have shown effectiveness, toxicity and other side effects have limited their usefulness. Inhibitors of hepatitis C virus (HCV) are useful to limit the establishment and progression of infection by HCV as well as in diagnostic assays for HCV.

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate.

Ledipasvir is described in U.S. Pat. No. 8,088,368 B2, and is a HCV NS5A inhibitor that has demonstrated potent anti-HCV activity against genotype (1a and 1b) HCV infection. Ledipasvir has the following chemical formula:

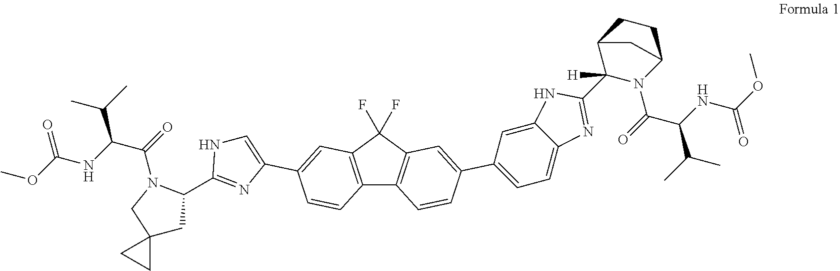 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

U.S. Pat. No. 9,056,860 B2 describes a process for the preparation of Ledipasvir wherein compound of formula (i) is reacted with compound of formula (ii) to obtain compound of formula (iii), further compound of formula (iii) is reacted with compound of formula (iv) to obtain Ledipasvir of formula 1, and the reaction scheme is as below:

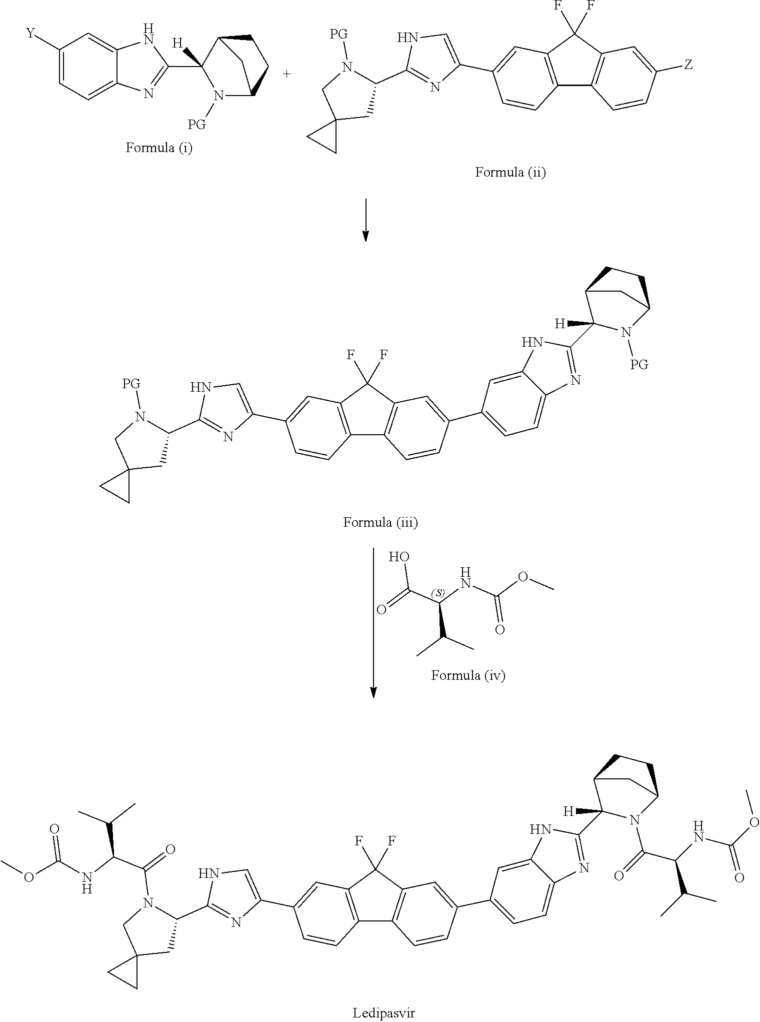

### SUMMARY OF THE INVENTION

In one aspect, the present invention relates to a process for the preparation of Ledipasvir of formula I and intermediates thereof.

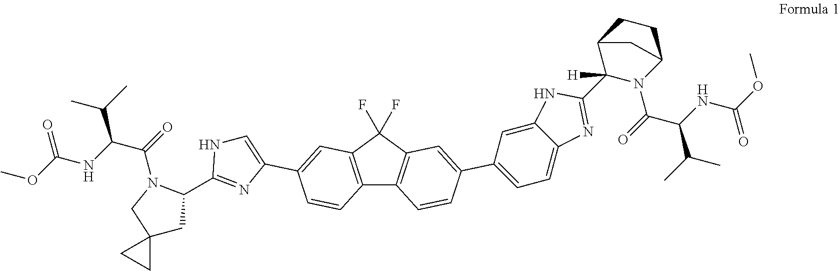 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

The process for preparation of ledipasvir and intermediates thereof according to present invention is described by reaction schemes-1, 2 & 3:

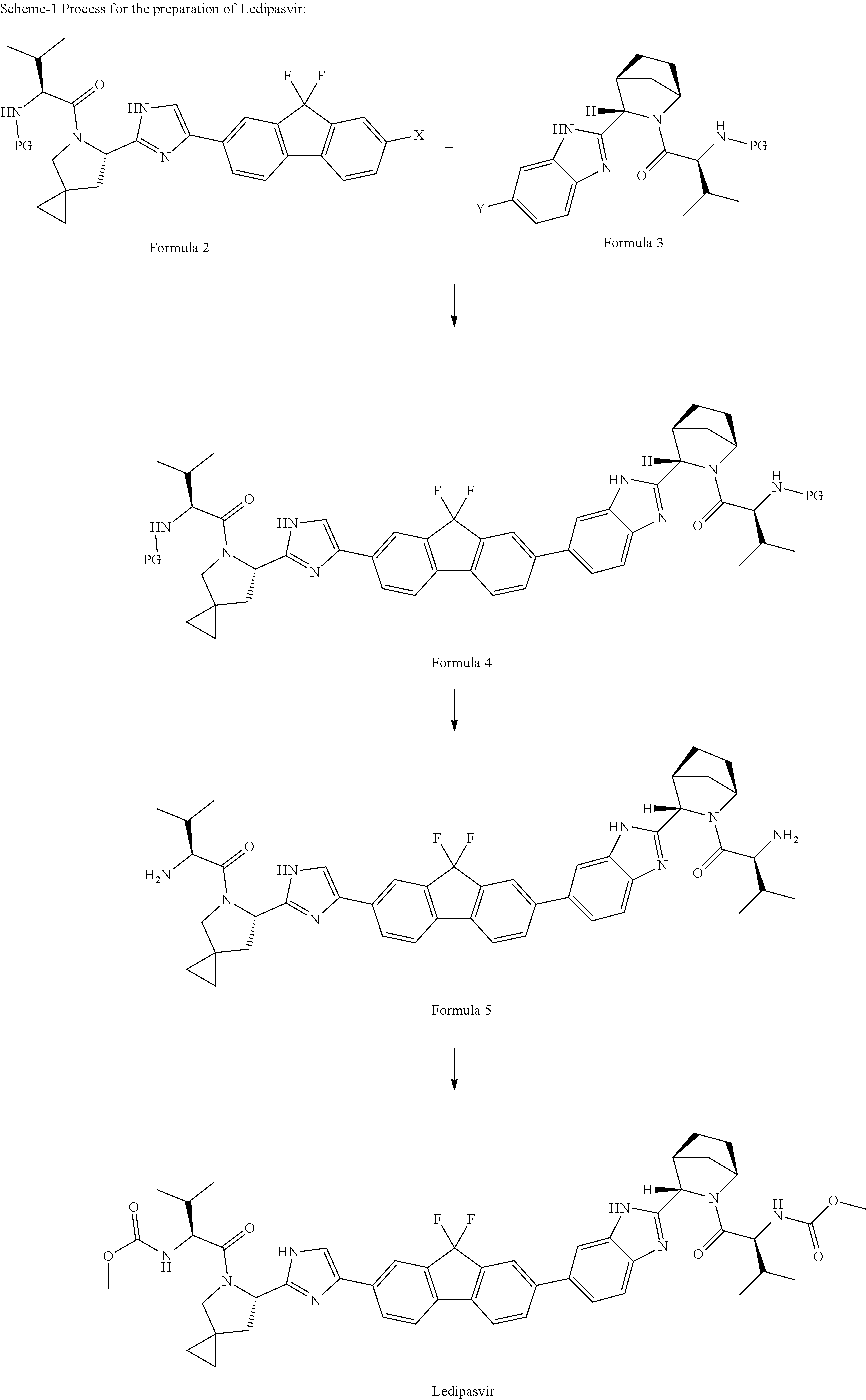

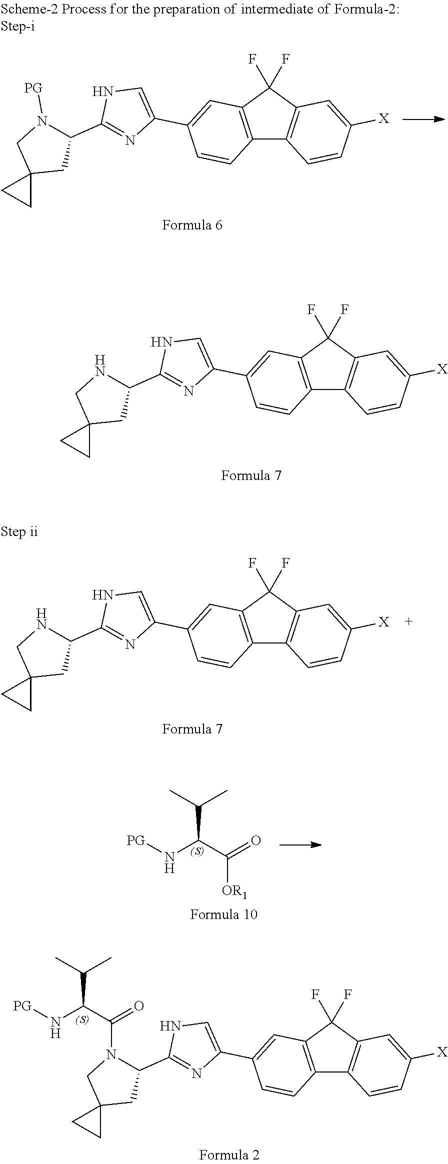

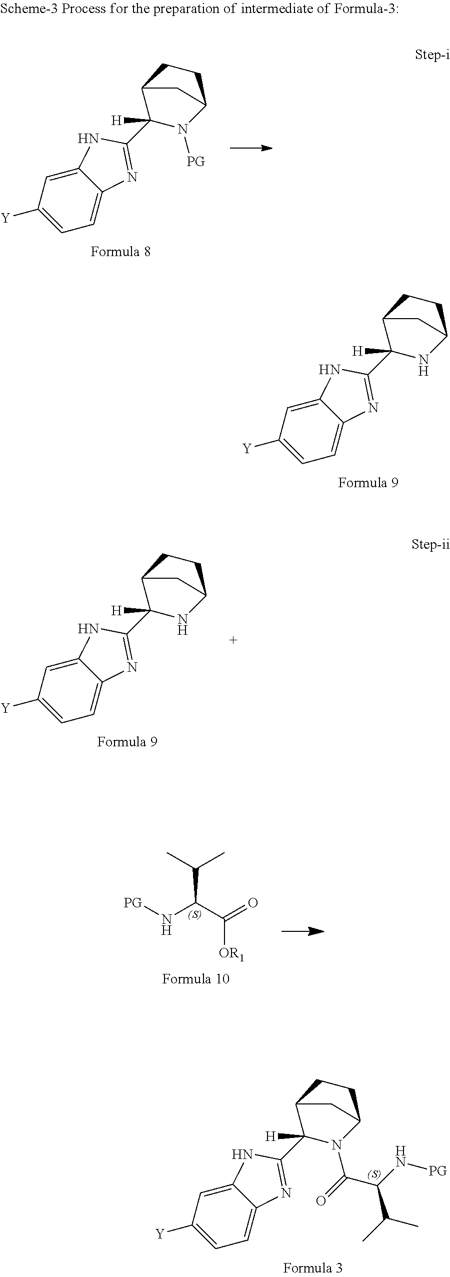

In another aspect, the present invention provides a novel intermediate of formula-2, formula-3, formula-4 and formula-5 or pharmaceutically acceptable salts, solvates thereof and process for the preparation thereof.

In another aspect, the invention provides use of novel intermediate of formula-2, formula-3, formula-4 and formula-5 or pharmaceutically acceptable salts, solvates thereof in the preparation of Ledipasvir.

In another aspect, the present invention relates to acetone solvate of compound of formula 4′ and process for the preparation thereof.

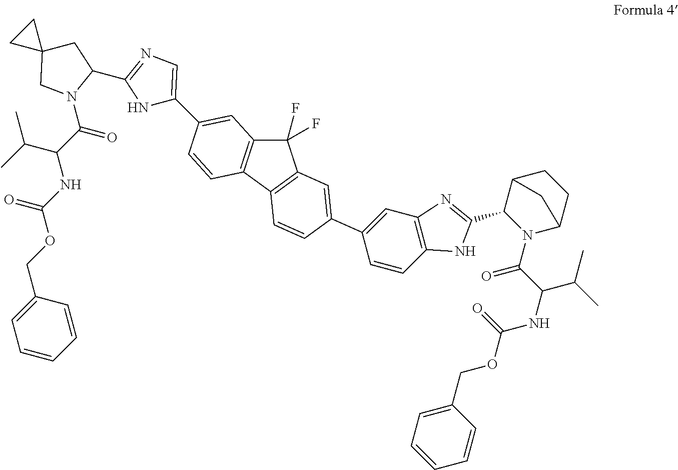 [SMILES: CC(C)C(NC(=O)OCc1ccccc1)C(=O)N1CC2(CC2)CC1c1ncc(-c2ccc3c(c2)C(F)(F)c2cc(-c4ccc5[nH]c([C@@H]6C7CCC(C7)N6C(=O)C(NC(=O)OCc6ccccc6)C(C)C)nc5c4)ccc2-3)[nH]1]

### DETAIL DESCRIPTION OF THE INVENTION

There is always a need for alternative preparative routes, which for example, use reagents, solvents that are less expensive, and/or easier to handle, consume smaller amounts of reagents and solvents, provide a higher yield of product, involve fewer steps, have smaller and/or more eco-friendly waste products, and/or provide a product of higher purity.

Each PG independently is an amine protecting group provided that amino protecting group is not carbomethyloxy (—COOCH₃) group. The term “amine protecting group” is well understood by the person skilled in synthetic organic chemistry as a moiety that can be selectively installed onto and removed from a suitable amine functional group. The field of protecting group methodology is advanced, and many amine protecting groups, and methods for using them, are well known in the art. The amine protecting group can be selected from tert-butyloxycarbonyl (BOC), Fluorenylmethyloxycarbonyl (F-MOC), N-benzyl, Trityl, substituted Carboxybenzyl (CBZ) group, etc.

Substituents X and Y are leaving groups and can be independently selected from any halogen, pseudo halogen, p-toluene sulfonate, methyl sulfonate, trifluoromethyl sulfonate, p-nitro benzene sulfonate and —B(OR)(OR′). In one embodiment, when Y is —B(OR)(OR′), then X is halogen or pseudohalogen, and in another embodiment, when Y is halogen or pseudohalogen, then X is —B(OR)(OR′).

The substituents R and R′ are independently selected from the group consisting of hydrogen and straight or branched C₁₋₈-alkyl, or R and R′ together represent a straight or branched C₁₋₈-alkylene, C₃₋₈-cycloalkylene, or C₆₋₁₂-arylene. Any alkyl, alkylene, cycloalkylene, or arylene as defined herein is optionally substituted with one or more substituents selected from the group consisting of C₁₋₆-alkyl, —C(O)N(C₁₋₆-alkyl)₂, and —C(O)O(C₁₋₆-alkyl).

The substituent R₁ is selected from hydrogen or hydroxy protecting group selected from straight or branched C₁₋₈-alkyl, substituted or unsubstituted aryl, heterocyclic aryl.

The halogen can be selected from chlorine, bromine, iodine or fluorine.

The pseudo-halogens are polyatomic halogen analogues can be selected from but not limited to, trifluoromethyl sulfonate, pentafluoro phenoxy.

The phrase “pharmaceutically acceptable salt” means a salt that is pharmaceutically acceptable. Examples of pharmaceutically acceptable salts include, but not limited to: (1) acid addition salts, formed with inorganic acids such as hydrochloric acid, hydrobromic acid, sulfuric acid, nitric acid, phosphoric acid, and the like; or formed with organic acids such as glycolic acid, pyruvic acid, lactic acid, malonic acid, malic acid, maleic acid, fumaric acid, tartaric acid, citric acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulfuric acid, gluconic acid, glutamic acid, salicylic acid, muconic acid, and the like or (2) basic addition salts formed with the conjugate bases of any of the inorganic acids listed above, wherein the conjugate bases comprise a cationic component selected from among Na⁺, Mg²⁺, Ca²⁺, NH_{g}R′″_{4-g}⁺, in which R′″ is a C₁₋₃ alkyl and g is a number selected from among 0, 1, 2, 3, or 4. It should be understood that all references to pharmaceutically acceptable salts include solvent addition forms (solvates) or crystal forms (polymorphs) as defined herein, of the same acid addition salt.

According to one aspect, the present invention provides a process for the preparation of Ledipasvir of formula I:

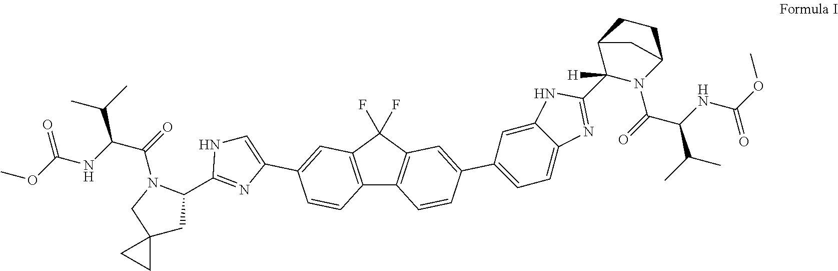 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

comprising the steps of:
i) reacting a compound of formula 2 or pharmaceutically acceptable salt thereof with a compound of formula 3 or pharmaceutically acceptable salt thereof; to obtain a compound of formula 4 or pharmaceutically acceptable salt thereof

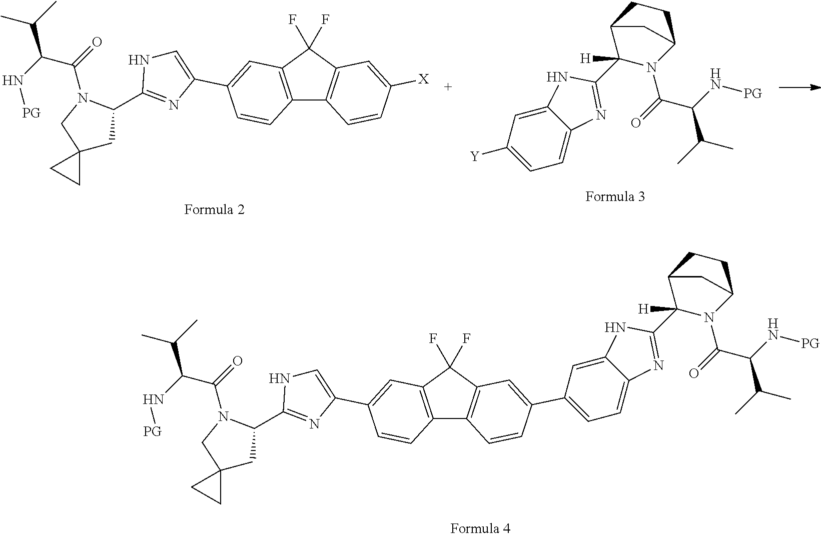

Wherein PG is amine protecting group;
ii) deprotecting the compound of formula 4 or the pharmaceutically acceptable salt thereof; to obtain a compound of formula 5 or pharmaceutically acceptable salt thereof:

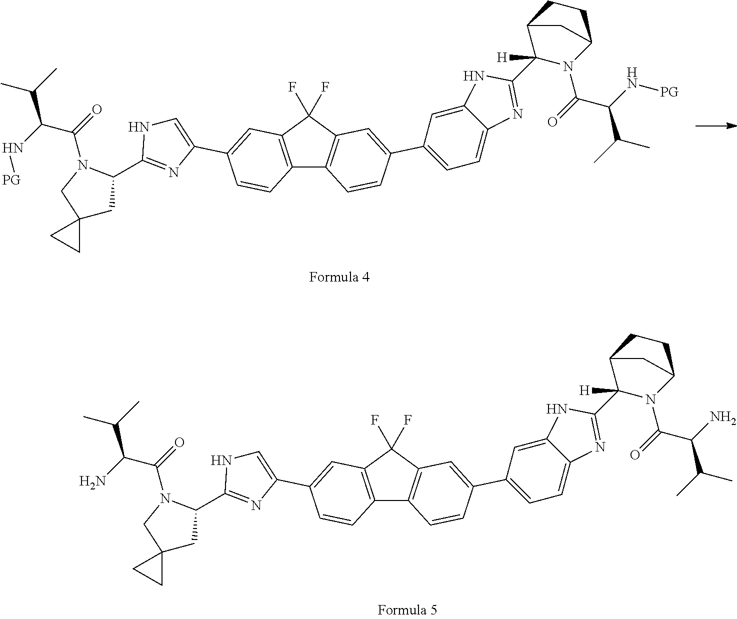

iii) converting compound of formula 5 or pharmaceutically acceptable salt thereof to Ledipasvir of formula 1.

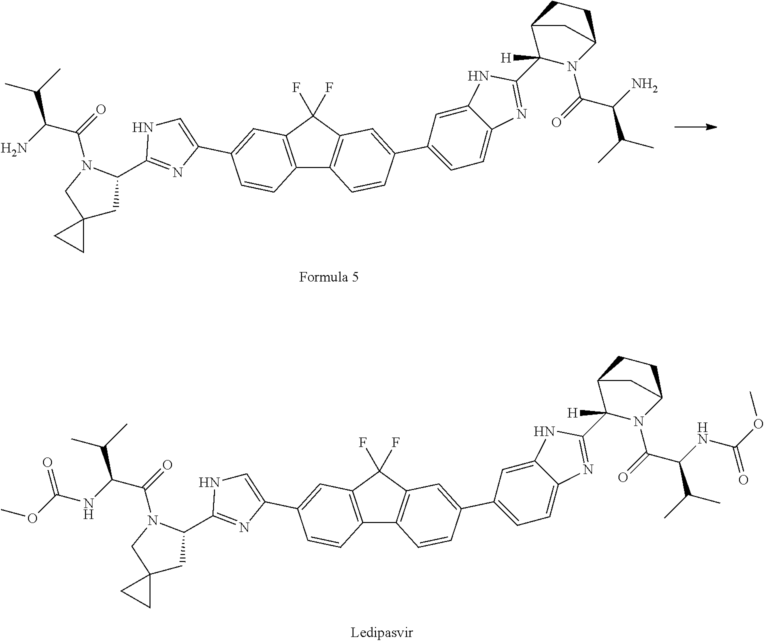

The reaction of Step-(i) can be performed in the presence of base like organic bases such as tertiary and secondary amines; inorganic bases such as sodium hydroxide, Potassium carbonate (K₂CO₃), sodium carbonate (Na₂CO₃), potassium hydroxide (KOH); salts thereof such as potassium fluoride, tripotassium phosphate (K₃PO₄) and the like.

The reaction of Step-(i) can optionally be performed in the presence of metal catalyst such as palladium, platinum, nickel, iron with or without ligands and salts thereof. The metal catalyst can be selected from Pd((PPh)₃)₄, PdCl₂(PPh)₃, As(PPh)₃, Pdcl₂[(Pt-Bu)₂Ph]₂, Me Phos [(2-dicylohexylphosphino 2-methyl biphenyl],Pd(OAc)₂, Pd(OAc)₂(PPh)₃, Pd(dba)₃,Pd(dppf)Cl₂, [Pd(dppb)Cl₂],Pd(dpa)₂,(dppf),Pd(dba)₃,Pd(2-Fur)₃,Pd(Pt-Bu)₃, [Pd(Joshiphos)Cl₂],Pd(PhCN)₂Cl₂.[PdCl2dppp] and the likes.

The solvent for the reaction of step-(i) can be selected from one or more of hydrocarbons like toluene, xylene; chlorinated hydrocarbons like methylene dichloride, ethylene dichloride and chlorobenzene; alcohols like methanol, ethanol; ethers like diethyl ether, diisopropyl ether, t-butyl methyl ether, 1,2-dimethoxy ether (DME), dibutyl ether, tetrahydrofuran, 1,4-dioxane; polar aprotic solvents like N,N-dimethylformamide, N,Ndimethyl acetamide, N-methylpyrrolidone, pyridine, dimethylsulfoxide, sulfolane, formamide, acetamide, propanamide, pyridine and acetonitrile or mixtures thereof. In particular, the solvent is methylene dichloride, 1,2-dimethoxy ether (DME), dimethylformamide, water, 1,4-dioxane, tetrahydrofuran, and acetonitrile or mixtures thereof.

Reaction of step-(ii) can be performed by maintaining the pH of the reaction to acidic by using reagents such as hydrogen halide in the solvent selected from one or more of hydrocarbons like toluene, xylene; chlorinated hydrocarbons like methylene dichloride, ethylene dichloride and chlorobenzene; alcohols like methanol, ethanol; ethers like diethyl ether, diisopropyl ether, t-butyl methyl ether, 1,2-dimethoxy ether (DME), dibutyl ether, tetrahydrofuran, 1,4-dioxane; polar aprotic solvents like N,N-dimethylformamide, N,Ndimethyl acetamide, N-methylpyrrolidone, dimethylsulfoxide, sulfolane, formamide, acetamide, propanamide, pyridine; acids like acetic acid and acetonitrile or mixtures thereof. In particular, the solvent is acetic acid, methylene dichloride, tetrahydrofuran, 1,2-dimethoxy ether (DME) and acetonitrile or mixtures thereof.

The reaction of step-(iii) can be performed in the presence of methyl chloroformate; methyl pentafluoro phenyl carbonate; dimethyl carbonate; anhydrides such as acetic anhydride, acetic formic anhydride and the like.

The reaction of step-(iii) can optionally be performed in the presence of base like organic bases such as tertiary and secondary amines; inorganic bases such as sodium hydroxide; potassium carbonate, sodium carbonate; potassium hydroxide.

The solvent for the reaction of step-(iii) can be selected from one or more of hydrocarbons like toluene, xylene; chlorinated hydrocarbons like methylene dichloride, ethylene dichloride and chlorobenzene; alcohols like methanol, ethanol; ethers like diethyl ether, diisopropyl ether, t-butyl methyl ether, dibutyl ether, tetrahydrofuran, 1,4-dioxane; polar aprotic solvents like N,N-dimethylformamide, N,N-dimethyl acetamide, N-methylpyrrolidone, pyridine, dimethylsulfoxide, sulfolane, formamide, acetamide, propanamide, pyridine and acetonitrile; water and/or mixtures thereof. In particular, the solvent is methylene dichloride, tetrahydrofuran, and acetonitrile or mixtures thereof.

In another aspect, the present invention provides a Ledipasvir having HPLC purity of more than 99.5%.

In another aspect, the present invention provides a novel intermediate of formula 2 or a pharmaceutically acceptable salt thereof.

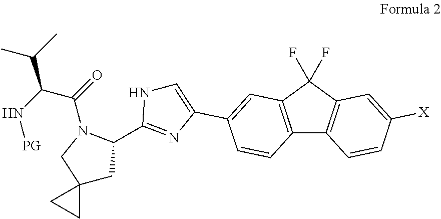

### Wherein:

PG is an amine protecting group; and X is leaving group.

In another aspect, the invention provides a process for the preparation of a novel intermediate of formula 2, comprising reaction of a compound of formula 7 with a compound of formula 10 in the presence of base like organic bases such as tertiary and secondary amines; inorganic bases such as sodium hydroxide, Potassium carbonate (K₂CO₃), sodium carbonate (Na₂CO₃), potassium hydroxide (KOH); salts thereof such as potassium fluoride, Tripotassium phosphate (K₃PO₄) and the like. Wherein X is a leaving group; R₁ is selected from hydrogen or hydroxy protecting group selected from straight or branched C1-8-alkyl, substituted or unsubstituted aryl, heterocyclic aryl, PG is an amine protecting group.

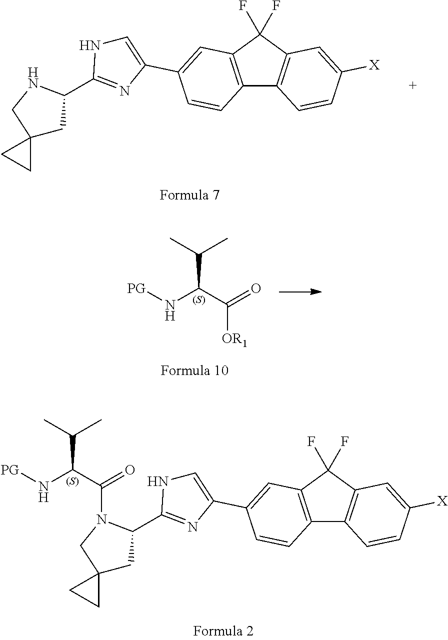

In another aspect, the present invention provides a novel intermediate of formula 3 or pharmaceutically acceptable salt or solvates thereof. Wherein PG is an amine protecting group and Y is a leaving group.

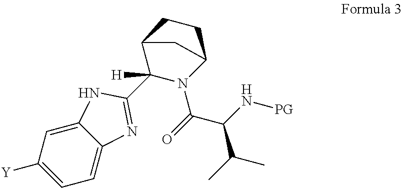

In another aspect, the invention provides a process for the preparation of a novel intermediate of formula 3; comprising reacting a compound of formula 9 with a compound of formula 10 in the presence of base like organic bases such as tertiary and secondary amines; inorganic bases such as sodium hydroxide, Potassium carbonate (K₂CO₃), sodium carbonate (Na₂CO₃), potassium hydroxide (KOH); salts thereof such as potassium fluoride, Tripotassium phosphate (K₃PO₄) and the like, wherein PG is an amine protecting group; R₁ is selected from hydrogen or hydroxy protecting group selected from straight or branched C1-8-alkyl, substituted or unsubstituted aryl, heterocyclic aryl and Y is a leaving group.

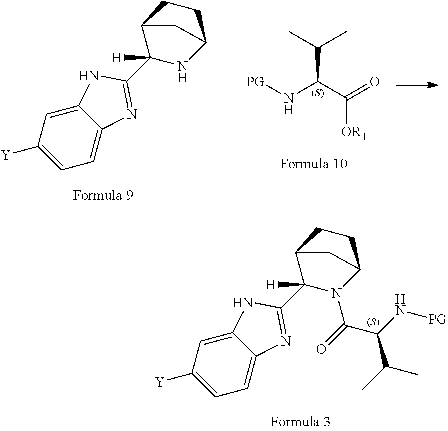

In another aspect, the present invention provides a novel intermediate of formula 4 or pharmaceutically acceptable salts or solvates thereof; wherein each PG an amine protecting group.

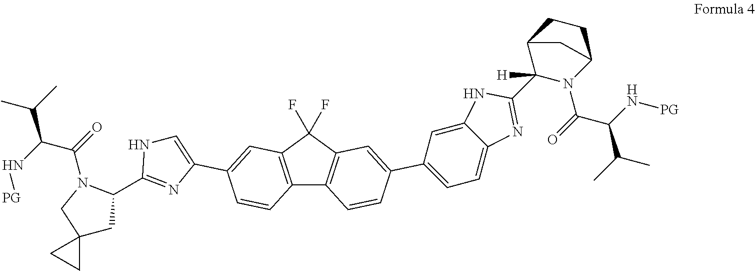

In another aspect, the invention provides a process for the preparation of a novel intermediate of formula 4 comprising reacting a compound of formula 2 or pharmaceutically acceptable salt thereof with a compound of formula 3 or pharmaceutically acceptable salt thereof in the presence of base like organic bases such as tertiary and secondary amines; inorganic bases such as sodium hydroxide, Potassium carbonate (K₂CO₃), sodium carbonate (Na₂CO₃), potassium hydroxide (KOH); salts thereof such as potassium fluoride, Tripotassium phosphate (K₃PO₄) and the like, wherein PG is an amine protecting group.

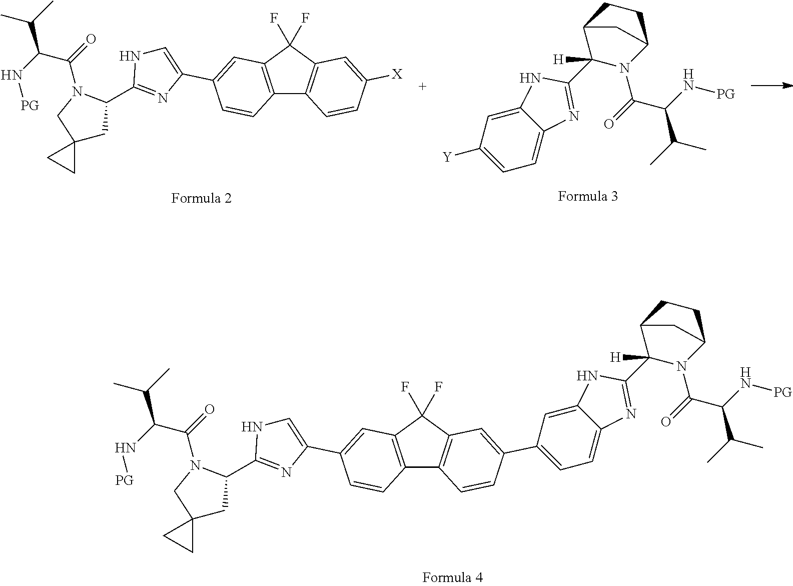

The process for preparation of compound of formula 4 can optionally be performed in presence of metal catalyst such as palladium, platinum, nickel, iron with or without ligands and salts thereof. The metal catalyst can be selected from Pd((PPh)₃)₄, PdCl₂(PPh)₃, As(PPh)₃, Pdcl₂[(Pt-Bu)₂Ph]₂, Me Phos[(2-dicylohexylphosphino 2-methyl biphenyl],Pd(OAc)₂, Pd(OAc)₂(PPh)₃, Pd(dba)₃,Pd(dppf)Cl₂ [Pd(dppb)Cl₂],Pd(dpa)₂,(dppf),Pd(dba)₃,Pd(2-Fur)₃,Pd(Pt-Bu)₃, [Pd(Joshiphos)Cl₂],Pd(PhCN)₂Cl₂. [PdCl2dppp] and the likes.

In another aspect, the present invention relates to an acetone solvate of compound of formula 4′.

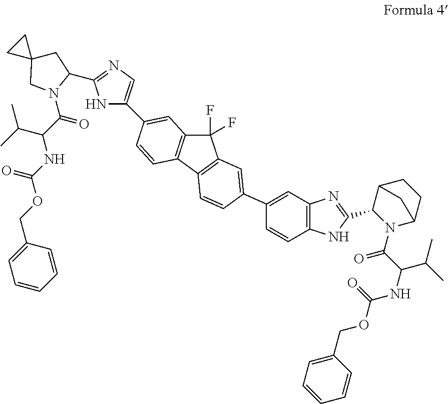 [SMILES: CC(C)C(NC(=O)OCc1ccccc1)C(=O)N1CC2(CC2)CC1c1ncc(-c2ccc3c(c2)C(F)(F)c2cc(-c4ccc5[nH]c([C@@H]6C7CCC(C7)N6C(=O)C(NC(=O)OCc6ccccc6)C(C)C)nc5c4)ccc2-3)[nH]1]

In another aspect, the invention provides use of acetone solvate of compound of formula 4′ in the preparation of Ledipasvir.

In another aspect, the invention provides a process for the preparation of an acetone solvate of compound of formula 4′ comprising heating the reaction mixture containing acetone and compound of formula 4′ to get clear solution, cooling the reaction mixture to room temperature to yield the acetone solvate of compound of formula 4′.

In another aspect, the present invention provides a novel intermediate of formula 5 or pharmaceutically acceptable salts or solvates thereof.

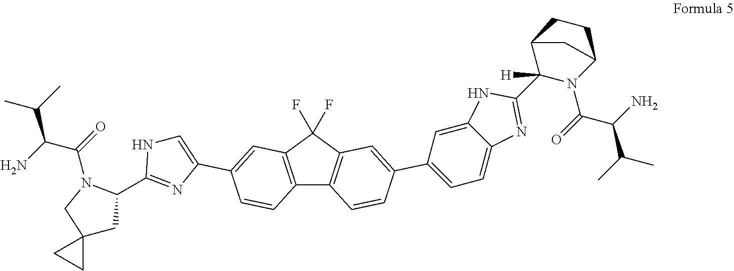 [SMILES: CC(C)[C@H](N)C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](N)C(C)C)[nH]c5c4)ccc2-3)c[nH]1]

In another aspect, the invention provides a process for the preparation of a novel intermediate of formula 5, comprising deprotecting a compound of formula 4 group by maintaining the pH of the reaction medium to acidic by using reagents such as hydrogen halide in a solvent, wherein PG is an amine protecting group.

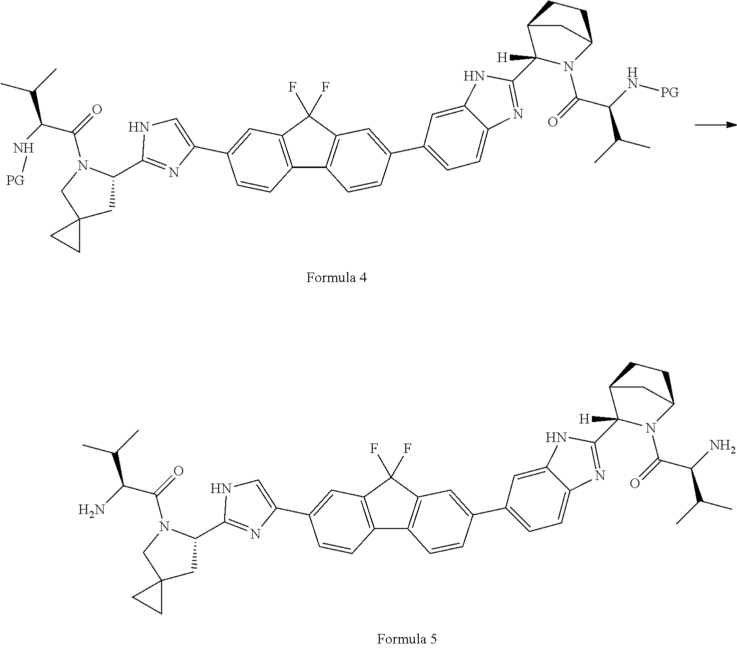

In another aspect, the present invention provides a process for preparation of ledipasvir comprising converting the compound of formula 5 or pharmaceutically acceptable salt thereof to Ledipasvir of formula 1, in the presence of methyl chloroformate; methyl pentafluoro phenyl carbonate; dimethyl carbonate; anhydrides such as acetic anhydride, acetic formic anhydride and the like.

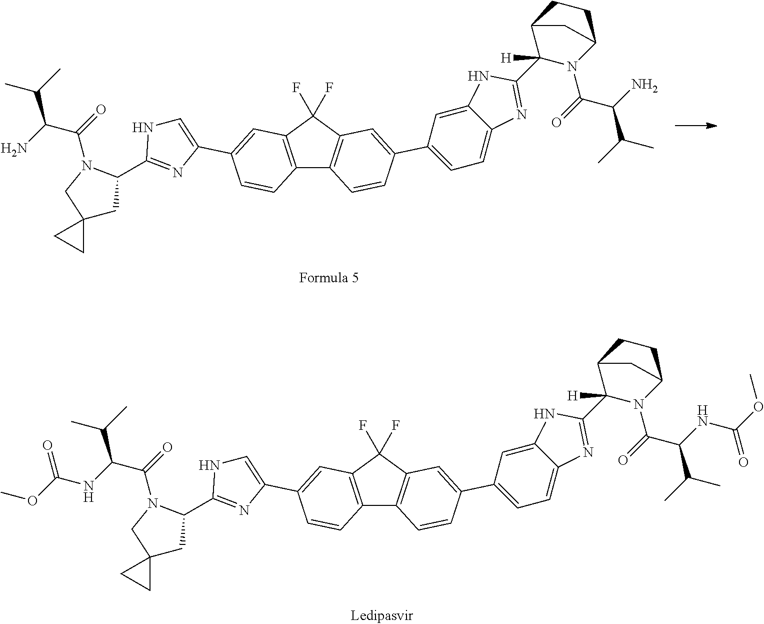

The conversion of compound of formula 5 or pharmaceutically acceptable salts thereof to ledipasvir can be done optionally in presence of base like organic bases such as tertiary and secondary amines; inorganic bases such as sodium hydroxide; potassium carbonate, sodium carbonate; potassium hydroxide.

Compounds of formula 8 & 6 can be synthesized by methods known in the literature via U.S. Pat. No. 9,056,860.

### EXAMPLES

### Example 1: Preparation of 2,5-dioxopyrrolidin-1-yl 2-(((benzyloxy)carbonyl)amino)-3-methylbutanoate (Formula 10′)

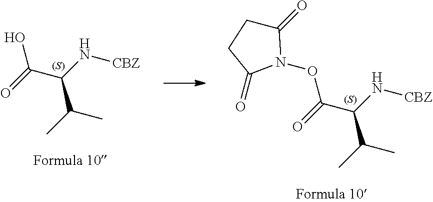

2-(((benzyloxy)carbonyl)amino)-3-methylbutanoic acid (65 g, 259 mMol), tetrahydrofuran (450 ml) were charged to a flask. N-hydroxy succinimide (33.3 g 285 mMol) was added to the reaction mass. Reaction mass was stirred for 10 mins. Dimethyl amino pyridine (DMAP, 1 g) was added to the reaction mass. Reaction mass was stirred and temperature of reaction mass was decreased to 5° C. Solution of DCC in THF (42.5%, 200 ml) was added dropwise to the reaction mass over a period of 45 minutes at 5° C. Reaction mass was stirred for 20 hours. Obtained solid was filtered; solvent was stripped off under reduced pressure. Solid was washed with heptane (3×250 ml), to get crude material (2,5-dioxopyrrolidin-1-yl 2-(((benzyloxy)carbonyl)amino)-3-methylbutanoate) with sufficient purity. Yield 85.5 g.

¹H NMR (DMSO-D6, 400 MHz): δ 1.02 (d, 6H), 2.24 (m, 1H), 2.82 (s, 4H), 4.37 (q, 1H), 5.09 (s, 2H), 7.34-7.44 (m, 5H), 8.09 (d, 1H).

¹³C NMR (DMSO D₆, 100 MHz): δ 18.3, 19.3, 25.4, 30.1, 58.1, 65.9, 127.7, 128.4, 136.7, 156.3, 168.0, 170.0, 172.8

FTIR (KBr): 3327, 2933, 2117, 1816, 1784, 1741, 1527, 1204, 893 cm⁻¹

MS (EI): C₁₇H₂₀N₂O₆ Exact mass: 348.35, observed mass: 366.2 (+H₂O).

### Example 2: Preparation of 6-(5-(7-bromo-9,9-difluoro-9H-fluoren-2-yl)-1H-imidazol-2-yl)-5-azaspiro[2.4]heptane hydrochloride (Formula 7′)

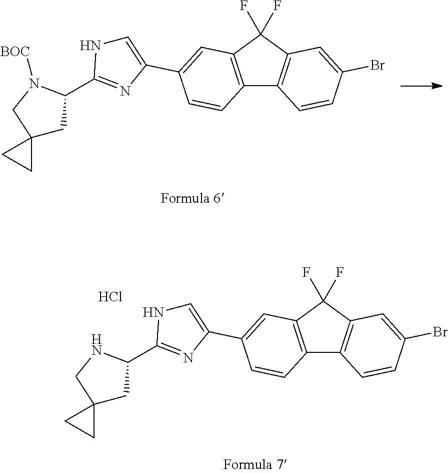 [SMILES: CC(C)(C)OC(=O)N1CC2(CC2)C[C@H]1c1nc(-c2ccc3c(c2)C(F)(F)c2cc(Br)ccc2-3)c[nH]1 ; FC1(F)c2cc(Br)ccc2-c2ccc(-c3c[nH]c([C@@H]4CC5(CC5)CN4)n3)cc21 ; Cl]

tert-butyl 6-(5-(7-bromo-9,9-difluoro-9H-fluoren-2-yl)-1H-imidazol-2-yl)-5-azaspiro[2.4]heptane-5-carboxylate (5 g, 9.2 mMol), Ethyl acetate (50 ml) were charged to flask. Solution of HCl in ethyl acetate (10%) was added dropwise to the reaction mass. The reaction mass was stirred for 2 hours at 55-60° C. Reaction mass was cooled to 20° C., filtered the solid 6-(5-(7-bromo-9,9-difluoro-9H-fluoren-2-yl)-1H-imidazol-2-yl)-5-azaspiro[2.4]heptane as HCl salt. Yield 4.6 g.

¹H NMR (MeOD, 400 MHz): δ 0.87 (s, 2H), 0.98 (s, 2H); 2.38 (m, 1H); 2.82 (m, 1H); 3.70 (d, 1H); 5.37 (1H), 7.42-8.15 (m, 7H).

FTIR (KBr): 3400, 2874, 1634, 1545, 1467, 1242, 1047, 821 cm⁻¹

MS (EI): C₂₂H₁₉BrClF₂N₃ Exact mass: 477.0 & 499.0 (Cl⁻) observed mass: 442.0 (without chloro pattern), 444.1 (with Bromo pattern)

### Example 3: Preparation of 2-((1S,3R,4R)-2-azabicyclo[2.2.1] hepatan-3-yl)-5-(4,4,5,5-tetramethyl-1,3,2-dioxaborolan-2-yl)-1H-benzo[d]imidazole hydrochloride (Formula 9′)

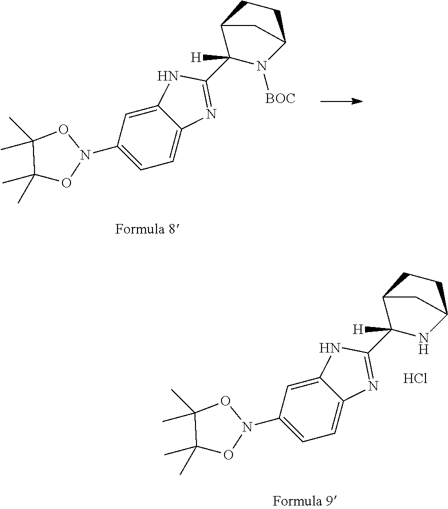 [SMILES: CC(C)(C)OC(=O)N1[C@@H]2CC[C@@H](C2)[C@H]1c1nc2ccc(N3OC(C)(C)C(C)(C)O3)cc2[nH]1 ; CC1(C)ON(c2ccc3nc([C@H]4N[C@@H]5CC[C@H]4C5)[nH]c3c2)OC1(C)C ; Cl]

1,4-dioxane (100 ml) was charged to flask. Flask was cooled to 0° C. Water (5 ml) was added to the flask. Oxalyl chloride (25 g) was added drop wise to the reaction mixture. Reaction mass was stirred for 30 minutes. (1S,3R,4R)-tert-butyl 3-(5-(4,4,5,5-tetramethyl-1,3,2-dioxaborolan-2-yl)-1H-benzo[d]imidazole-2-yl)-2-azabicyclo[2.2.1]heptane-2-carboxylate (10 g, 22.7 mMol) was added to reaction mass. Reaction mass was stirred for 30 minutes at 55-60° C. Ethyl acetate (10 ml) was added to the reaction mass. Reaction mass was stirred for 30 minutes at 0-5° C. Obtained solid was filtered under vacuum. Solid was dried overnight under vacuum to give 2-((1S,3R,4R)-2-azabicyclo[2.2.1] hepatan-3-yl)-5-(4,4,5,5-tetramethyl-1,3,2-dioxaborolan-2-yl)-1H-benzo[d]imidazole hydrochloride salt. Yield 7.4 g

¹H NMR (MeOD, 400 MHz): δ 1.39 (s, 12H), 1.85-2.11 (m, 5H), 2.27 (d, 1H), 3.32 (s, 1H), 4.36 (s, 1H), 5.16 (1H), 7.87 (d, 1H), 7.96 (d, 1H), 8.20 (s, 1H)

¹³C NMR (MeOD 100 MHz): δ 25.2, 26.1, 27.9, 37.7, 41.0, 58.2, 61.5, 85.7, 114.5, 121.8, 132.4, 133.7, 134.7, 147.8

FTIR (KBr): 3437, 2976, 1623, 1360, 1145, 855 cm⁻¹

MS (EI): C₁₉H₂₇BClN₃O₂ exact mass: 375.7 observed mass: 340.2 (without chloro counter ion)

### Example 4: Preparation of 2-((1S,3R,4R)-2-azabicyclo[2.2.1]heptan-3-yl)-5-(4,4,5,5-tetramethyl-1,3,2-dioxaborolan-2-yl)-1H-benzo[d]imidazole hydrochloride (Formula 9′)

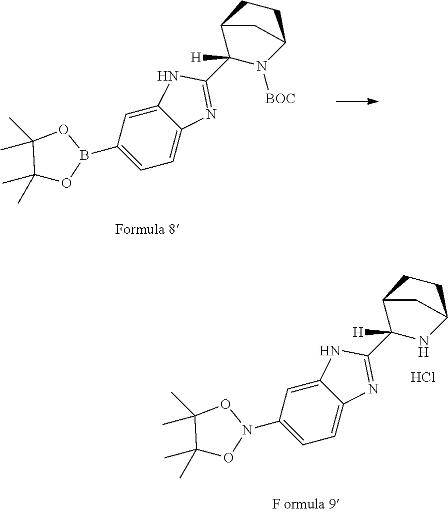 [SMILES: CC(C)(C)OC(=O)N1[C@@H]2CC[C@@H](C2)[C@H]1c1nc2ccc(B3OC(C)(C)C(C)(C)O3)cc2[nH]1 ; CC1(C)ON(c2ccc3nc([C@H]4N[C@@H]5CC[C@H]4C5)[nH]c3c2)OC1(C)C ; Cl]

(1S, 3R, 4R)-tert-butyl 3-(5-(4,4,5,5-tetramethyl-1,3,2-dioxaborolan-2-yl)-1H-benzo[d]imidazole-2yl)-2-azabicyclo[2.2.1] heptane-2-carboxylate (50 g, 114 mMol) was added to a flask. Ethyl acetate (250 ml) was added to the flask. 10% Solution of hydrochloric acid and ethyl acetate (250 ml) was added drop-wise to reaction mixture over a period of 30 minutes. Reaction mass was stirred for 2.0 hours at 55-60° C. Reaction mass was cooled to 20° C. Obtained solid was filtered under vacuum. Solid was dried overnight under vacuum to give 2-((1S,3R,4R)-2-azabicyclo[2.2.1]heptan-3-yl)-5-(4,4,5,5-tetramethyl-1,3,2-dioxaborolan-2-yl)-1H-benzo[d]imidazole hydrochloride salt. Yield 44.1 g.

### Example 5: Preparation of benzyl ((S)-1-((S)-6-(4-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate (Formula 2′)

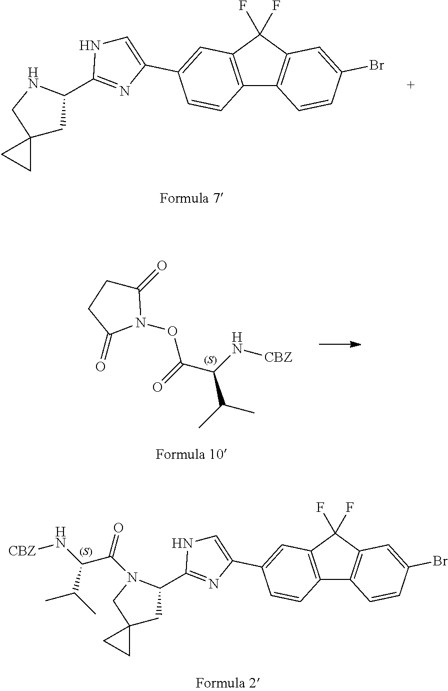

6-(5-(7-bromo-9,9-difluoro-9H-fluoren-2-yl)-1H-imidazol-2-yl)-5-azaspiro[2.4]heptane as HCl salt (22 g, 46.0 mMol) was charged in flask. Ethyl acetate (220 ml) was added to flask. Potassium carbonate (7 g) was added to the reaction mass. Reaction mass was stirred for 70 minutes at 55-60° C. Solution of (2,5-dioxopyrrolidin-1-yl-2-(((benzyloxy)carbonyl)amino)-3-methylbutanoate) (20.1 g, 57.5 mMol) was added to reaction mass at 55-60° C. Reaction mass was stirred for 4 hours. Reaction was monitored using silica gel TLC. After completion of reaction, solvent was evaporated under reduced pressure. Crude product benzyl ((S)-1-((S)-6-(4-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate was purified by passing through silica gel column. Yield 13.6 g

FTIR: 3392, 2962, 1715, 1628, 1455, 1244, 1052, 823, 697 cm⁻¹

¹⁹F NMR (MeOD, 376 MHz): δ 112.09 & 111.92 ppm

Mass (EZ): C₃₅H₃₃BrF₂N₄O₃ exact mass 674.17 & 676.17. Observed mass 675.3 & 677.2 (bromo pattern).

### Example 6: Preparation of benzyl (3-methyl-1-oxo-1-((1S,3R,4R)-3-(5-(4,4,5,5-tetramethyl-1,3,2-dioxaborolan-2-yl)-1H-benzo[d]imidazole-2-yl)-2-azabicyclo[2.2.1]heptan-2-yl)butan-2-yl)carbamate (Formula 3′)

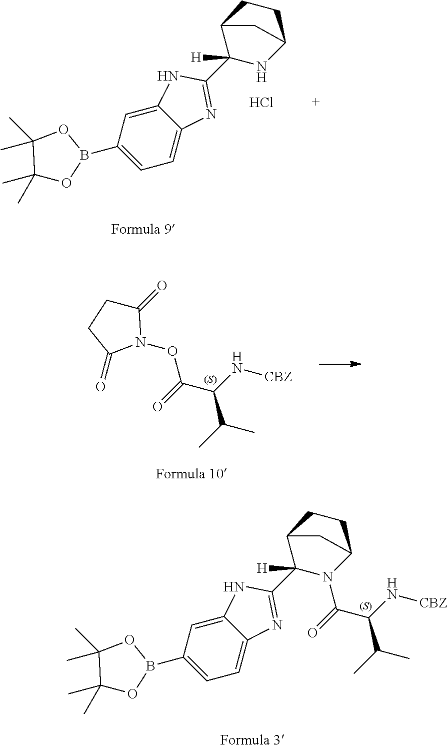

2-((1 S,3R,4R)-2-azabicyclo[2.2.1] heptan-3-yl)-5-(4,4,5,5-tetramethyl-1,3,2-dioxaborolan-2-yl)-1H-benz[d] imidazole hydrochloride salt (18.7 g 375.5 mMol) was charged to flask. Water (40 ml) was charged to the flask. Solution potassium carbonate (30%, 40 ml) was added dropwise to the reaction mixture. Reaction mass was stirred for 45 min at 25-30° C. Reaction mass was extracted with ethyl acetate (3×60 ml). Solution of (2,5-dioxopyrrolidin-1-yl 2-(((benzyloxy)carbonyl)amino)-3-methylbutanoate) (16.6 g, 47.5 mMol) was added to the reaction mass. Reaction mass was stirred for 3.5 hrs. Reaction was monitored using silica gel TLC. After completion of reaction, solvent was evaporated under reduced pressure. Crude product (benzyl (3-methyl-1-oxo-1-((1 S,3R,4R)-3-(5-(4,4,5,5-tetramethyl-1,3,2-dioxaborolan-2-yl)-1H-benzo[d]imidazole-2-yl)-2-azabicyclo[2.2.1]heptan-2-yl)butan-2-yl)carbamate) was purified by passing through silica gel column chromatography. Yield 19.2 g.

¹H NMR (MeOD, 400 MHz): δ 0.82-1.06 (M, 8H), 1.21 (S, 9H), 1.34 (S, 3H), 2.03 (S, 2H), 2.79 (S, 2H), 4.04-4.13 (M, 1H), 4.31-4.35 (M, 1H), 4.66-4.86 (M, 1H), 5.09-5.16 (D, 3H), 5.44 (d, 1H), 7.31-7.39 (M, 8H)

MS (EI): C₃₂H₄₂BN₄O₅ exact mass: 572.32, Observed mass: 573.2 (m+1)

### Example 7: Preparation of benzyl (1-(6-(5-(7-(2-((3S)-2-(((benzyloxy)carbonyl)valyl)-2-azabicyclo[2.2.1]heptan-3-yl)-1H-benzo[d]imidazol-5-yl)-9,9-difluoro-9H-fluoren-2-yl)-1H-imidazol-2-yl)-5-azaspiro[2.4]heptan-5-yl)-3-methyl-1-oxobutan-2-yl)carbamate (Formula 4′)

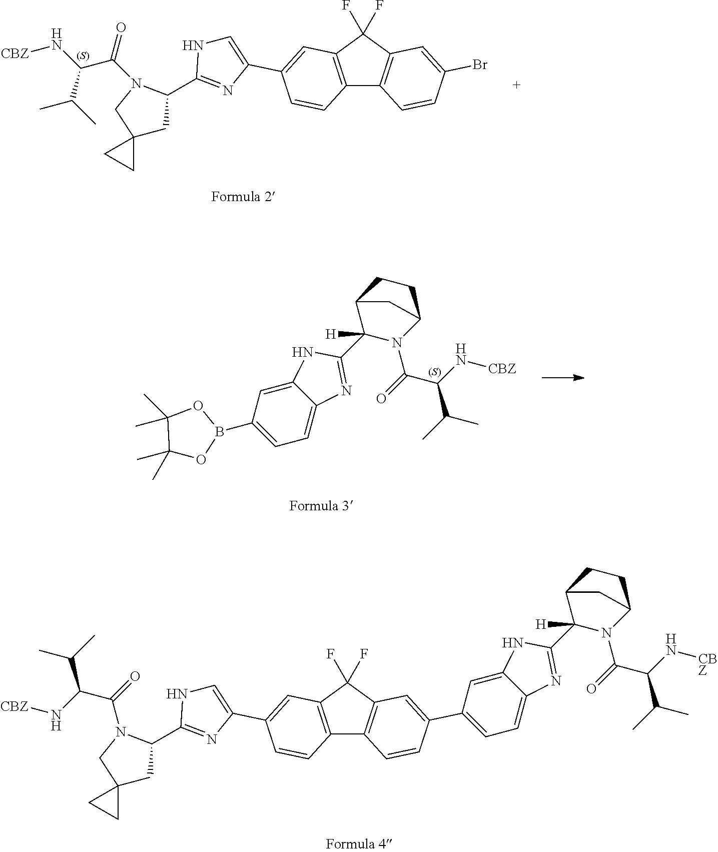

Potassium carbonate (8.8 g, 63.9 mMol) was charged to flask. Water (34 ml) was added to the flask. benzyl ((S)-1-((S)-6-(4-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate (14.4 g, 21.3 mMol) was charged to flask. Benzyl (3-methyl-1-oxo-1-((1S,3R,4R)-3-(5-(4,4,5,5-tetramethyl-1,3,2-dioxaborolan-2-yl)-1H-benzo[d]imidazole-2-yl)-2-azabicyclo[2.2.1]heptan-2-yl)butan-2-yl)carbamate) (11.0 g, 19.2 mMol) was added to reaction mass. DME (420 ml) was added to reaction mass. Reaction mass was stirred for 30 min at 55-60° C. Pd(PPh3)₄ (2.5 g, 2.1 mMol) was added to reaction mass at 55-60° C. Reaction mass was stirred for 4.0 hours. (benzyl (3-methyl-1-oxo-1-((1S,3R,4R)-3-(5-(4,4,5,5-tetramethyl-1,3,2-dioxaborolan-2-yl)-1H-benzo[d]imidazole-2-yl)-2-azabicyclo[2.2.1]heptan-2-yl)butan-2-yl)carbamate) (4.8 g, 8.4 mMol) was added to reaction mass. Reaction was monitored using silica gel TLC. After completion of reaction, solvent was evaporated under reduced pressure. Crude benzyl (1-(6-(5-(7-(2-((3S)-2-(((benzyloxy)carbonyl)valyl)-2-azabicyclo[2.2.1]heptan-3-yl)-1H-benzo[d]imidazol-5-yl)-9,9-difluoro-9H-fluoren-2-yl)-1H-imidazol-2-yl)-5-azaspiro[2.4]heptan-5-yl)-3-methyl-1-oxobutan-2-yl)carbamate was purified by passing through silica gel column chromatography (eluent Ethyl acetate/Hexane). Yield 11.3 g.

¹⁹F NMR (MeOD, 376 MHz): δ−111.82-111.77

FTIR (KBr): 3282, 2962, 1714, 1629, 1515, 1440, 1232, 1041, 696 cm⁻¹

MS (EI): C₆₁H₆₂F₂N₈O₆ Exact mass: 1041.19 Observed Mass: 1042.1 (+1)

### Example 8: Preparation of 2-amino-1-(6-(5-(7-(2-((3S)-2-(2-amino-3-methylbutanoyl)-2-azabicyclo[2.2.1]heptan-3-yl)-1H-benzo[d]imidazole-5-yl)-9,9-difluoro-9H-fluoren-2-yl)-1H-imidazol-2-yl)-5-azaspiro[2.4]heptan-5-yl)-3-methylbutan-1-one (Formula 5′)

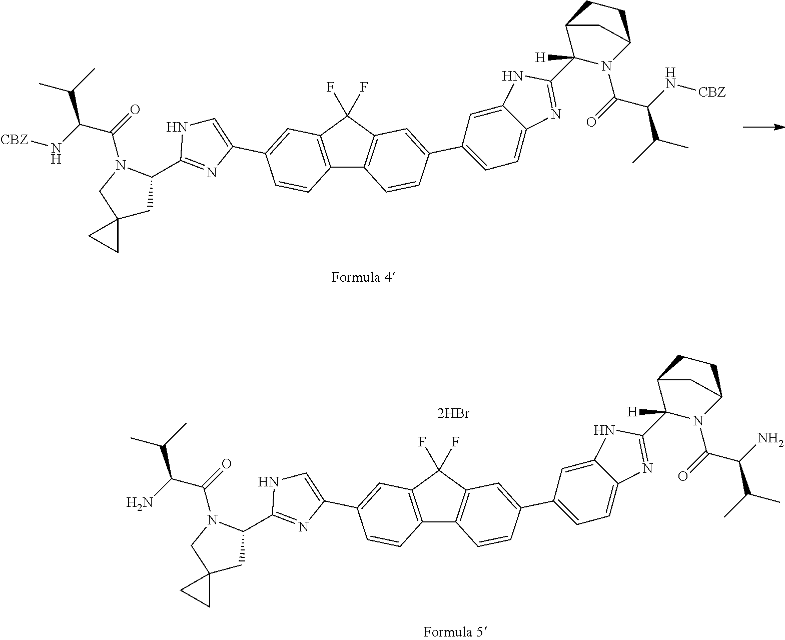

benzyl (1-(6-(5-(7-(2-((3S)-2-(((benzyloxy)carbonyl)valyl)-2-azabicyclo[2.2.1]heptan-3-yl)-1H-benzo[d]imidazol-5-yl)-9,9-difluoro-9H-fluoren-2-yl)-1H-imidazol-2-yl)-5-azaspiro[2.4]heptan-5-yl)-3-methyl-1-oxobutan-2-yl)carbamate (12.5 g, 12.1 mMol) was charged to flask. Acetic acid (12.5 ml) was added to the flask. Solution of hydrogen bromide in acetic acid (30%, 26 ml 96.6 mMol) was added dropwise to the reaction mass at 20-25° C. Reaction mass was stirred for 3.5 hours at 25-30° C. DME (200 ml) was added to the reaction mass under vigorous stirring. Reaction mass was stirred for 1 hour to precipitate out salt. Solid was filtered under vacuum and washed with DME (100 ml). Solid was dried under vacuum for 12 hours to give 2-amino-1-(6-(5-(7-(2-((3S)-2-(2-amino-3-methylbutanoyl)-2-azabicyclo[2.2.1]heptan-3-yl)-1H-benzo[d] imidazole-5-yl)-9,9-difluoro-9H-fluoren-2-yl)-1H-imidazol-2-yl)-5-azaspiro[2.4]heptan-5-yl)-3-methylbutan-1-one as HBr salt. Yield 11.7 g.

¹⁹F NMR (MeOD, 376 MHz): δ−112.29

MS (EI): C₄₅H₅₂Br₂F₂N₈O₂ exact mass: 934.25 observed mass: 773.6 (+1; without bromide counter ion).

FTIR (KBr): 3411, 2966, 1642, 1454, 1054, 817 CM⁻¹

### Example 9: Preparation of 2-amino-1-(6-(5-(7-(2-((3S)-2-(2-amino-3-methylbutanoyl)-2-azabicyclo[2.2.1]heptan-3-yl)-1H-benzo[d]imidazole-5-yl)-9,9-difluoro-9H-fluoren-2-yl)-1H-imidazol-2-yl)-5-azaspiro[2.4]heptan-5-yl)-3-methylbutan-1-one (Formula 5′)

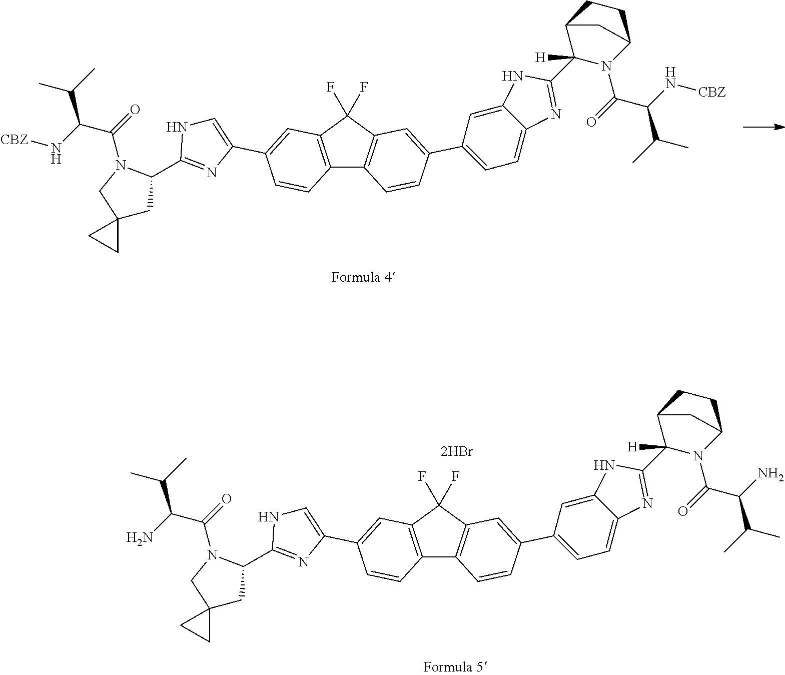

benzyl (1-(6-(5-(7-(2-((3S)-2-(((benzyloxy)carbonyl)valyl)-2-azabicyclo[2.2.1]heptan-3-yl)-1H-benzo[d]imidazol-5-yl)-9,9-difluoro-9H-fluoren-2-yl)-1H-imidazol-2-yl)-5-azaspiro[2.4]heptan-5-yl)-3-methyl-1-oxobutan-2-yl)carbamate (50 gm, 0.0489 moles) and dichloromethane (200 ml) were added to a flask. Solution of Trimethylsilyl iodide (42.2 gm; 0.211 moles) in dichloromethane (100 ml) were added to the reaction flask. Reaction mass was stirred and after completion of reaction, the reaction mass was added slowly to water (500 ml). Aqueous layer was separated and treated with Aq.KOH solution till pH reaches in between 8-9. The solid was filtered and washed with water and dried to afford 2-amino-1-(6-(5-(7-(2-((3S)-2-(2-amino-3-methylbutanoyl)-2-azabicyclo[2.2.1]heptan-3-yl)-1H-benzo[d]imidazole-5-yl)-9,9-difluoro-9H-fluoren-2-yl)-1H-imidazol-2-yl)-5-azaspiro[2.4]heptan-5-yl)-3-methylbutan-1-one (35 gm).

### Example 10: Preparation of Ledipasvir

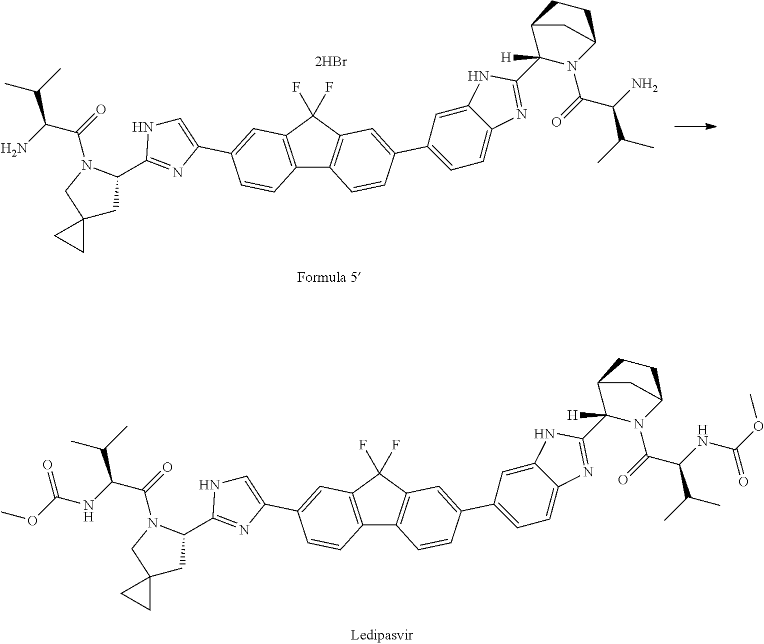

2-amino-1-(6-(5-(7-(2-((3S)-2-(2-amino-3-methylbutanoyl)-2-azabicyclo[2.2.1]heptan-3-yl)-1H-benzo[d]imidazole-5-yl)-9,9-difluoro-9H-fluoren-2-yl)-1H-imidazol-2-yl)-5-azaspiro[2.4]heptan-5-yl)-3-methylbutan-1-one hydrobromide (7 gm; 0.009 moles), dichloromethane (100 ml) were added to flask. Reaction mass was stirred and trimethylamine (3.66 gm; 0.036 moles) were added to the reaction mass. Solution of sodium carbonate (1.92 m; 0.018 moles) in water (21 ml) was added to the reaction mass. The reaction mass was stirred and cooled to 0 to −30° C. methyl pentafluorophenyl carbonate (7.2 gm; 0.0299 moles) was slowly added to the reaction mass. Reaction mass was stirred and after completion of reaction, aq.potassium hydroxide (5%) was slowly added to the reaction mass. Layers were separated. Organic layer was concentrated under vacuum, at 35° C. solid was isolated by crystallization from mixture of acetone and acetonitrile, isolated solid was dissolved in methanol. The reaction mass was slowly added to water at 25-35° C. and stirred. The slurry was filtered off and washed with water. Solid was dried under vacuum at 40-45° C. to afford Ledipasvir (6 gm).

### Example 11: Preparation of Ledipasvir

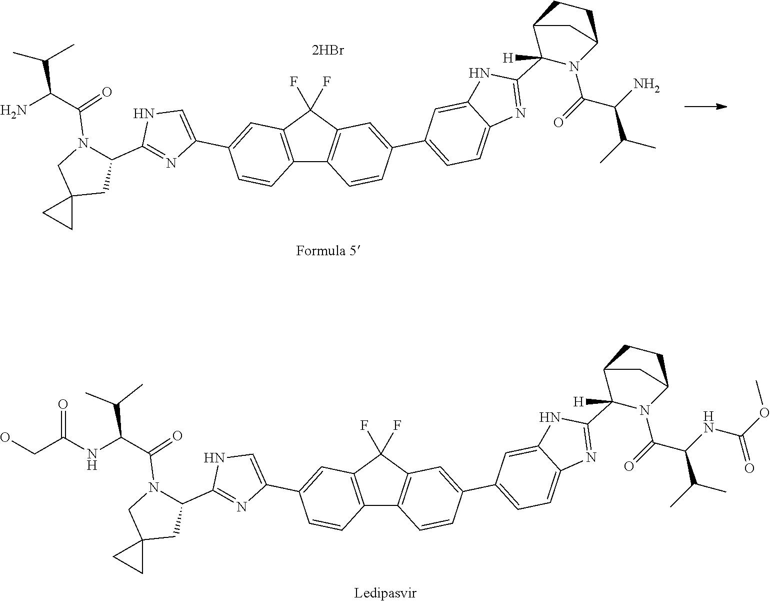 [SMILES: CC(C)[C@H](N)C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](N)C(C)C)[nH]c5c4)ccc2-3)c[nH]1 ; COC(=O)N[C@H](C(=O)N1[C@@H]2CC[C@@H](C2)[C@H]1c1nc2ccc(-c3ccc4c(c3)C(F)(F)c3cc(-c5c[nH]c([C@@H]6CC7(CC7)CN6C(=O)[C@@H](NC(=O)CO)C(C)C)n5)ccc3-4)cc2[nH]1)C(C)C]

2-amino-1-(6-(5-(7-(2-((3S)-2-(2-amino-3-methylbutanoyl)-2-azabicyclo[2.2.1]heptan-3-yl)-1H-benzo[d]imidazole-5-yl)-9,9-difluoro-9H-fluoren-2-yl)-1H-imidazol-2-yl)-5-azaspiro[2.4]heptan-5-yl)-3-methylbutan-1-one as HBr salt (10 g, 10.7 mMol and DCM (100 ml) was charged to flask. Triethylamine (9 ml) was added to reaction mass. Methyl chloroformate (2.2, 23.4 mMol) was added dropwise to the reaction mass at 0-5° C. Reaction mass was stirred for 3.5 hours at 5-30° C. reaction mass was quenched using Methyl amine solution (2 ml) and brine (20%, 50 ml). Reaction mass was stirred for 15 min, layer were separated. Solvent was distilled out under vacuum to give crude product. Crude product was purified using column chromatography to get pure Ledipasvir. Yield 2.87 g (HPLC purity: 99.58%)

MS (EI): C₄₉H₅₄F₂N₈O₆ Exact mass: 889.0 Observed mass: 889.4 (M+)

¹⁹F NMR (MeOD, 376 MHz): δ−111.7 & −111.64

FTIR (KBr): 3294, 2962, 1719, 1633, 1516, 1447, 1238, 1041, 776 cm⁻¹

### Example 12: Preparation of benzyl (3-methyl-1-oxo-1-((1S,3R,4R)-3-(5-(4,4,5,5-tetramethyl-1,3,2-dioxaborolan-2-yl)-1H-benzo[d]imidazol-2-yl)-2-azabicyclo[2.2.1]heptan-2-yl)carbamate (Formula 3′)

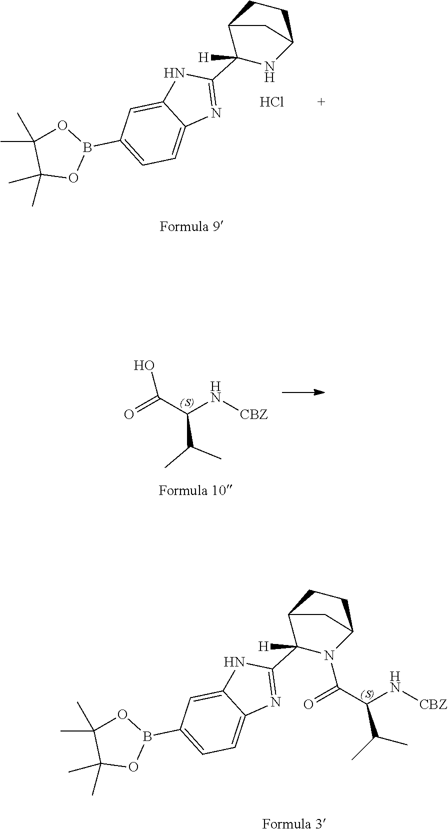

2-(((benzyloxy)carbonyl)amino)-3-methylbutanoic acid (18 g, 71.7 mMol) was added to a flask. Dimethyl formamide (50 ml) was added to the flask. 2-((1S,3R,4R)2azabicyclo[2.2.1] heptan-3-yl)-5-(4,4,5,5-tetramethyl-1,3,2-dioxaborolan-2-yl-1H-benzo[d]imidazole hydrochloride salt (26.9 g, 71.7 mMol) was added to the reaction mass. N,N-Diisopropylethylamine (40 ml) was added to reaction mass. 1-[Bis(dimethylamino)methylene]-1H-1,2,3-triazolo[4,5-b]pyridinium 3-oxide hexafluorophosphate (HATU, 27 g, 71.7 mMol) was added to the reaction mass at 0-5° C. Reaction mass was stirred for 100 minutes at 25-30° C. Water (500 ml) was added to the reaction mass to form scummy mass, afterwards water was decanted from the mass. Water (200 ml) was added to the reaction mass, reaction mass was extracted with dichloromethane (3×80 ml). Crude product benzyl (3-methyl-1-oxo-1-((1S,3R,4R)-3-(5-(4,4,5,5-tetramethyl-1,3,2-dioxaborolan-2-yl)-1H-benzo[d]imidazol-2-yl)-2-azabicyclo[2.2.1]heptan-2-yl)carbamate purified by passing through silica gel column. Crude weight 45.0 g. 29 g crude material is passed through column to give 22.5 g pure product.

¹¹B NMR (MeOD, 128 MHz): δ 33.27

MS (EI): C₁₉H₂₇BClN₃O₂ Exact mass: 375.7 Observed mass: 340.2 (without chloride counter ion)

### Example 13: Preparation of benzyl(1-(6-(5-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate (Formula 2′)

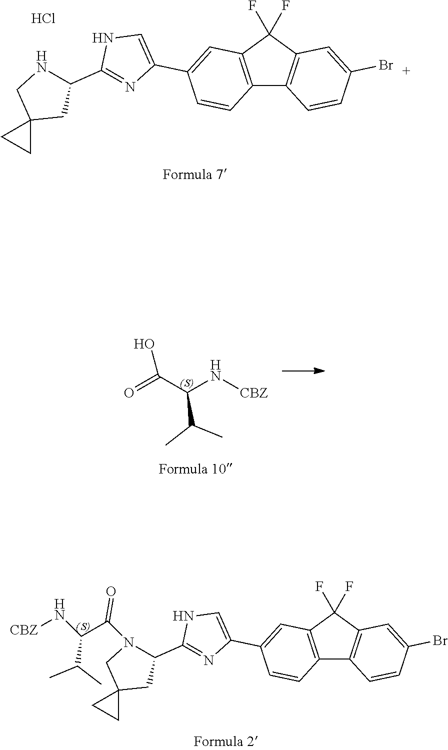

2-(((benzyloxy)carbonyl)amino)-3-methylbutanoic acid (11 g, 43.9 mMol) to the flask. Dimethyl formamide (85 ml) was added to the flask. (6-(5-(7-bromo-9,9-difluoro-9H-fluoren-2-yl)-1H-imidazol-2-yl)-5-azaspiro[2.4]heptane as hydrochloride salt (21 g, 43.9 mMol) was added to the flask. N,N-Diisopropylethylamine (42 ml) was added to the reaction mass. 1-[Bis(dimethylamino)methylene]-1H-1,2,3-triazolo[4,5-b]pyridinium 3-oxide hexafluorophosphate (HATU, 16.7 g, 43.9 mMol) was added to the reaction mass at 0-5° C. Water (500 ml) was added to the reaction mass to form scummy mass, afterwards water was decanted from the mass. Water (400 ml) was added to the reaction mass, reaction mass was extracted with dichloromethane (3×80 ml). Crude product (benzyl(1-(6-(5-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate) crude weight 35.0 g.

### Example 14 Preparation of an Acetone Solvate of Compound of Formula 4′

Potassium carbonate (8.8 g) was charged to flask. Water (34 ml) was added to the flask. benzyl (1-(6-(5-(7-bromo-9,9-difluoro-9H-fluoren-2-yl)-1H-imidazol-2-yl)-5-azaspiro[2.4]heptan-5-yl)-3-methyl-1-oxobutan-2-yl)carbamate (14.4 gg) was charged to flask. (benzyl (3-methyl-1-oxo-1-((1S,3R,4R)-3-(5-(4,4,5,5-tetramethyl-1,3,2-dioxaborolan-2-yl)-1H-benzo[d]imidazole-2-yl)-2-azabicyclo[2.2.1]heptan-2-yl)butan-2-yl)carbamate) 11 g) was added to reaction mass. N-propanol (130 ml) was added to reaction mass. Reaction mass was stirred for 30 min at 60-65° C. Pd(PPh3)₄ (2.5 g) was added to reaction mass at 60-65° C. Reaction mass was stirred for 4.0 hours. Reaction was monitored using silica gel TLC. After completion of reaction, reaction mass was cooled. Solvent was removed. Toluene (85 ml) was added to reaction mass at 50-60° C. Reaction mass was stirred and cooled. Water (85 ml) was added to reaction mass, layers were separated, toluene was distilled out. Acetone (88 ml) was added to the reaction mass at 50-60° C. Reaction mass was cooled and stirred. solid was filtered, washed with acetone and dried. Yield 11.3 g (65-85%).

1: A process for the preparation of Ledipasvir, comprising:
i) reacting a compound of formula 2 or pharmaceutically acceptable salt thereof with a compound of formula 3 or pharmaceutically acceptable salts thereof to yield compound of formula 4: 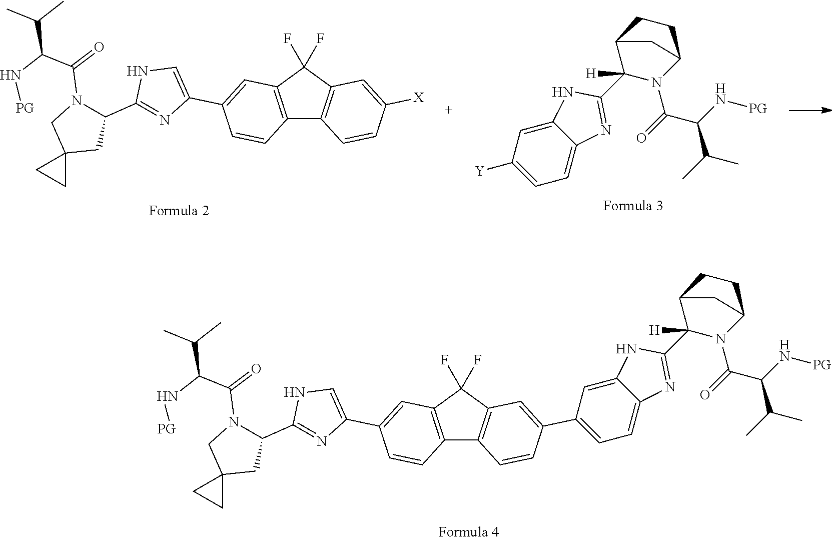
wherein PG is amine protecting group provided that amino protecting group is not carbomethyloxy (—COOCH₃) group; X and Y are leaving groups;
ii) deprotecting a compound of formula 4 or pharmaceutically acceptable salts in acidic medium to yield a compound of formula 5 or pharmaceutically acceptable salts; and 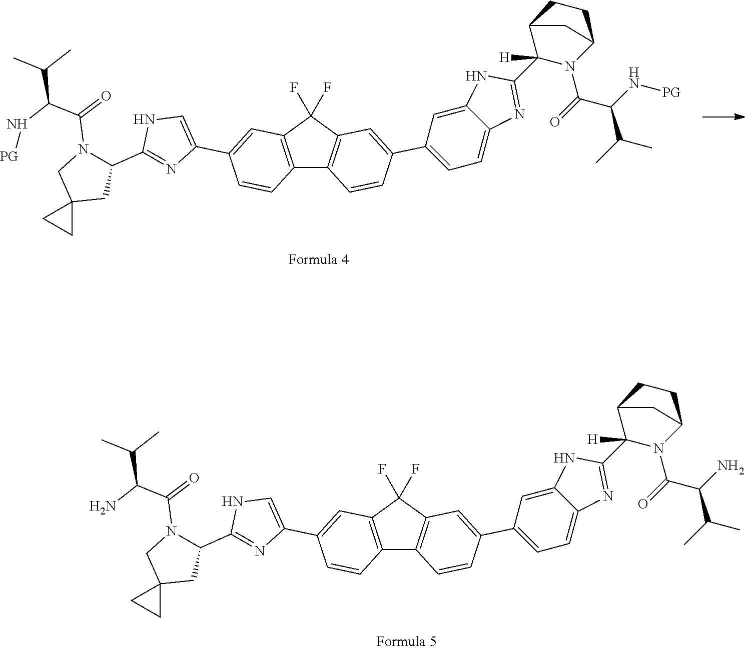
iii) converting compound of formula 5 or pharmaceutically acceptable salt thereof to ledipasvir.

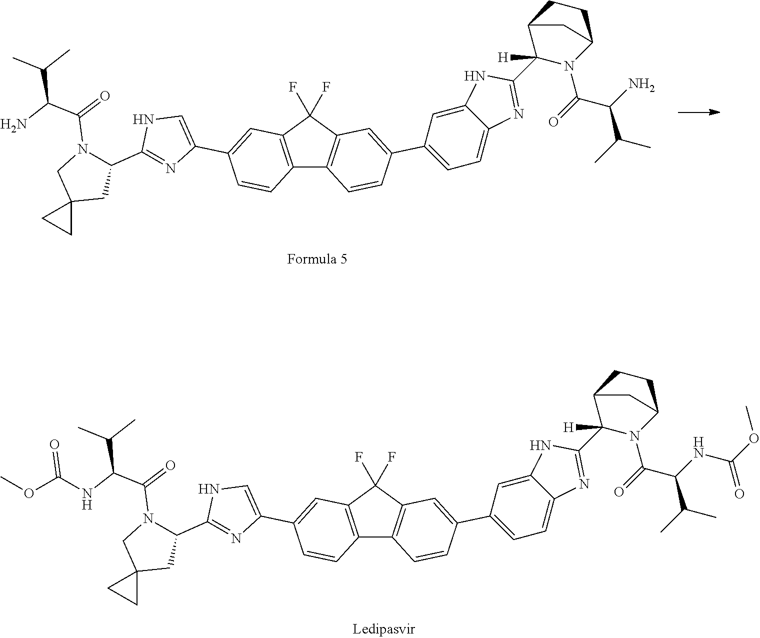

2-24. (canceled)

25: The process according to claim 1 wherein the step (i) is carried in presence of base and/or metal catalyst selected from palladium, platinum, nickel, iron with or without ligands and salts thereof.

26: The process according to claim 1, wherein the step (iii) is performed in the presence of methyl chloroformate; methyl pentafluoro phenyl carbonate; dimethyl carbonate; acetic anhydride or acetic formic anhydrides.

27: A compound of formula 2 or its pharmaceutically acceptable salts or solvates thereof 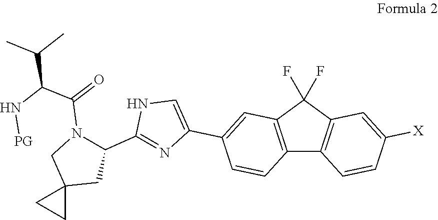
wherein PG is an amine protecting group provided that amino protecting group is not carbomethyloxy (—COOCH₃) group; X is a leaving group.

28: A process for the preparation of compound of formula 2 comprising reaction of compound of formula 7 with a compound of formula 10 in presence of base: 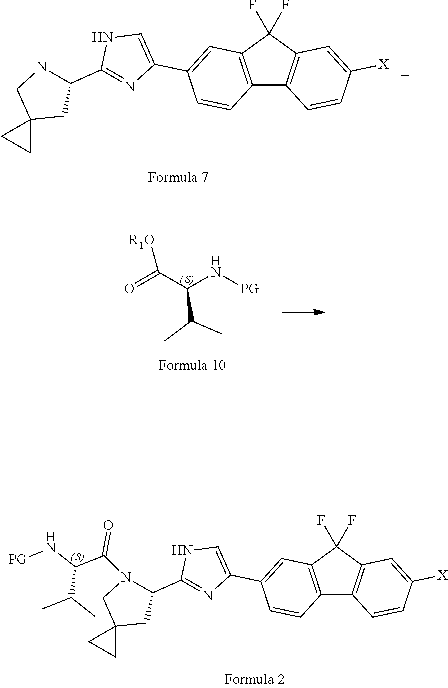
wherein X is a leaving group; R₁ is hydrogen or hydroxyl protecting group; PG is an amine protecting group provided that amino protecting group is not carbomethyloxy (—COOCH₃) group.

29: A compound of formula 3 or pharmaceutically acceptable salts or solvates thereof: 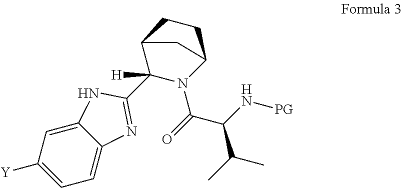
wherein X is a leaving group; PG is amine protecting group provided that amino protecting group is not carbomethyloxy (—COOCH₃) group.

30: A process for the preparation of compound of formula 3 comprising reaction of compound of formula 9 with a compound of formula 10 in the presence of base: 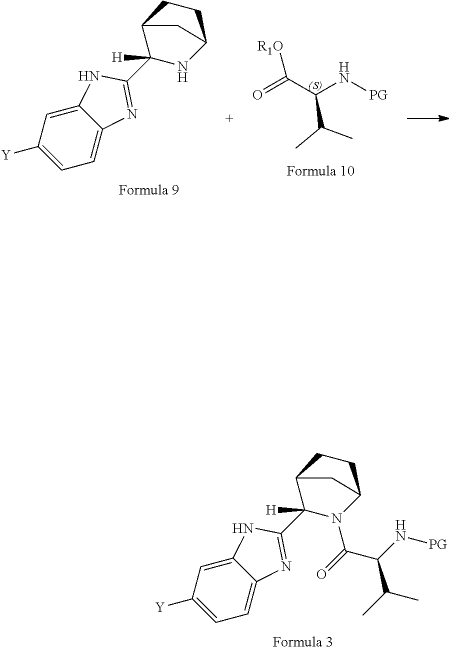
wherein Y is a leaving group; R₁ is hydrogen or hydroxyl protecting group; PG is an amine protecting group provided that amino protecting group is not carbomethyloxy (—COOCH₃) group.

31: A compound of formula 4 or pharmaceutically acceptable salts or solvates thereof 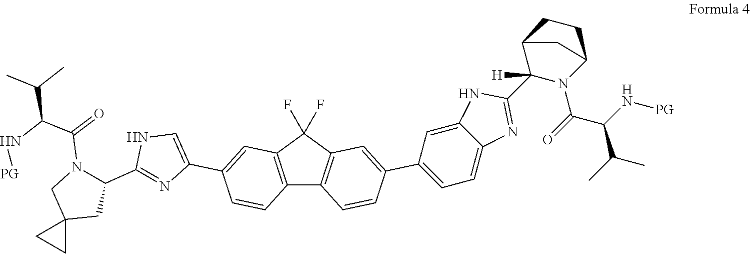
wherein PG is an amine protecting group provided that amino protecting group is not carbomethyloxy (—COOCH₃) group.

32: A process for the preparation of compound of formula 4 comprising reaction of compound of formula 2 with a compound of formula 3 in the presence of base and/or metal catalyst: 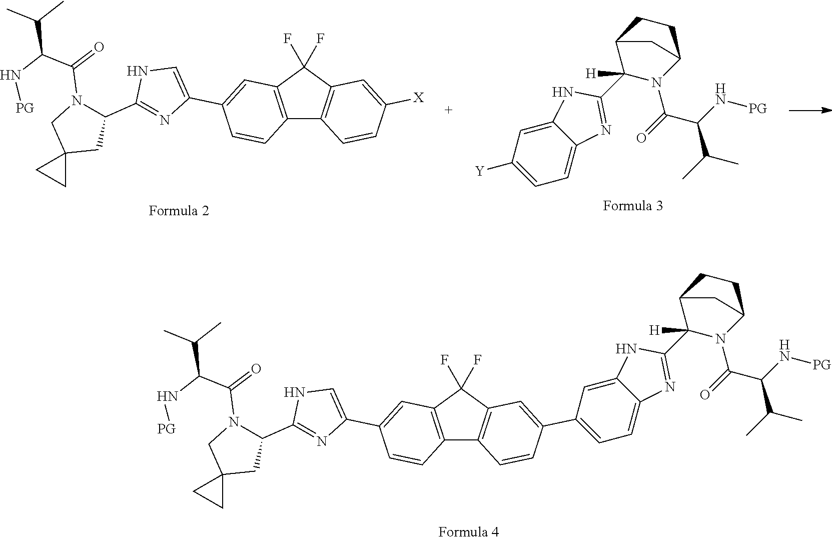
wherein X and Y are leaving groups; PG is an amine protecting group provided that amino protecting group is not carbomethyloxy (—COOCH₃) group.

33: A compound of formula 5 or pharmaceutically acceptable salts or solvates thereof.

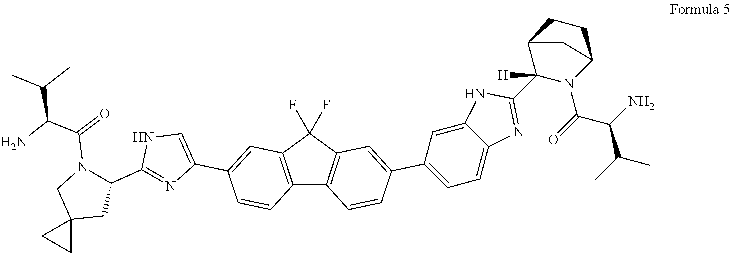 [SMILES: CC(C)[C@H](N)C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](N)C(C)C)[nH]c5c4)ccc2-3)c[nH]1]

34: A process for the preparation of compound of formula 5 comprising deprotection of a compound of formula 4 in acidic medium; 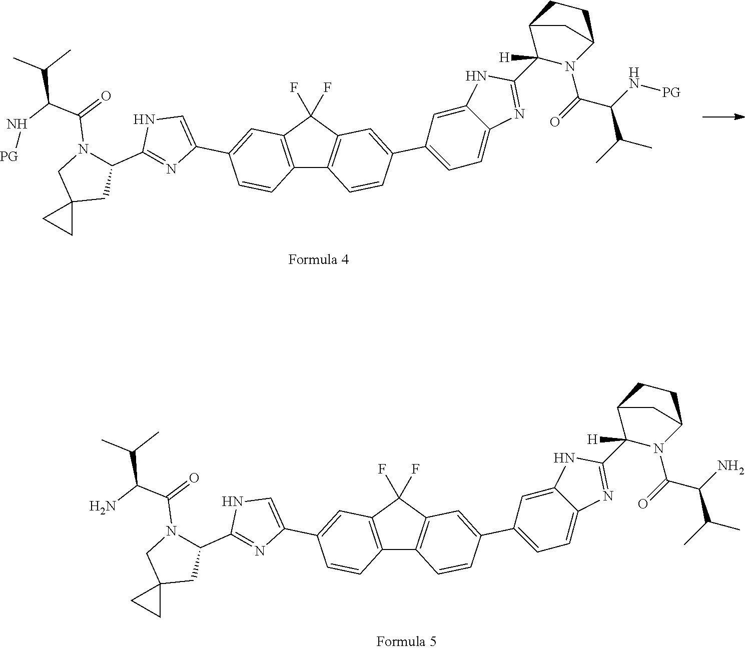
wherein PG is an amine protecting group provided that amino protecting group is not carbomethyloxy (—COOCH₃) group, and its conversion to Ledipasvir.

35: A process according to claim 34 wherein the conversion to Ledipasvir is performed in the presence of methyl chloroformate; methyl pentafluoro phenyl carbonate; dimethyl carbonate; acetic anhydride or acetic formic anhydrides.

36: An acetone solvate of compound of formula 4′.

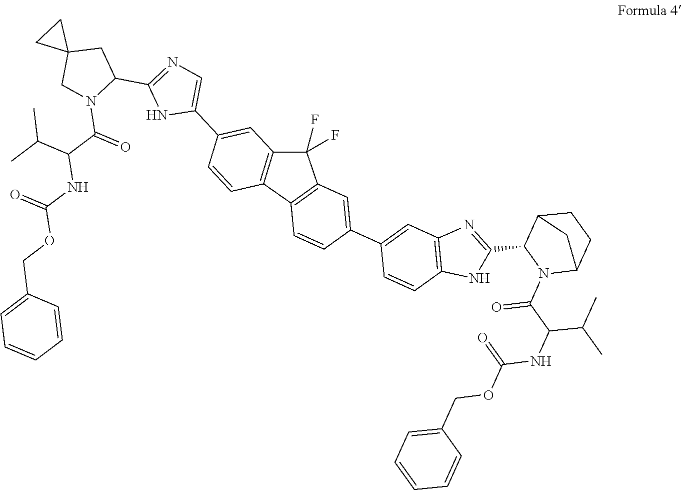 [SMILES: CC(C)C(NC(=O)OCc1ccccc1)C(=O)N1CC2(CC2)CC1c1ncc(-c2ccc3c(c2)C(F)(F)c2cc(-c4ccc5[nH]c([C@@H]6C7CCC(C7)N6C(=O)C(NC(=O)OCc6ccccc6)C(C)C)nc5c4)ccc2-3)[nH]1]

37: A process for preparation of an acetone solvate of compound of formula 4′ comprising heating the reaction mixture containing acetone and compound of formula 4′ to get clear solution, cooling the reaction mixture to room temperature to yield the acetone solvate of compound of formula 4′.

38: Use of an acetone solvate of compound of formula 4′ as claimed in claim 36 in the further preparation of Ledipasvir.
